(19) United States
(12) Patent Application Publication
(10) Pub. No.: US 2021/0077217 A1
(43) Pub. Date: Mar. 18, 2021

(54) Pin Closure System

(71) Applicant: Klarity Medical Products, LLC, Newark, OH (US)

(72) Inventor: Matthew P Calhoun, Newark, OH (US)

(21) Appl. No.: 16/568,792

(22) Filed: Sep. 12, 2019

(51) Int. Cl. A61B 90/14; A61N 5/10; F16B 19/02; F16B 21/16

(52) U.S. Cl. CPC A61B 90/14; A61N 5/1001; A61N 2005/1097; F16B 21/16; F16B 19/02

(57) ABSTRACT

A pin closure system, especially for use in securing the upper and lower frame plates of a radiation therapy treatment mask. An upper sleeve having an expansile portion is placed and engaged within a shaped fenestration of an upper frame plate, while a lower sleeve having an expansile portion is placed and engaged within a shaped fenestration of a lower frame plate. An axial pin is inserted spanning both sleeves, causing the expansile portions to expand radially, engaging the sleeves together and locking the plates together. The system may also be used to use a single pin to simultaneously lock the upper and lower frame plates together and to an external substrate, such as a treatment table for repeatable and highly accurate patient positioning.

### CROSS-REFERENCE TO RELATED APPLICATIONS

Not applicable.

### STATEMENT REGARDING FEDERALLY SPONSORED RESEARCH OR DEVELOPMENT

Not applicable.

### TECHNICAL FIELD

The present disclosure relates generally to a pin closure system, particularly to a system for securing an upper plate to a lower plate and further particularly to securing the upper frame plate to the lower frame plate of a radiation therapy mask, well-suited for patient immobilization during radiotherapy or other applications requiring a high degree of accuracy in patient positioning. This is of particular importance for radiotherapy of the head and neck region.

### BACKGROUND OF THE INVENTION

The present invention relates to a pin closure system, for securing an upper and lower plate, and particularly for securing the upper and lower frame plates of a radiation therapy positioning device, or in any application for persons whose bodies or body parts are required to be retained in a particular position or attitude. In particular, a need exists for a simple and easy to use immobilization system that allows repetitive patient positioning with a high degree of accurate repeatability across time, for example, wherein radiation treatments must be directed at a certain anatomical point across different times. Such precise positioning of the head and neck area, as might be required for radiotherapy of that region, is particularly problematic due to difficulty in immobilizing the area with a reasonable degree of patient comfort, while at the same time having the immobilization be easily adjustable and removable.

In particular, accurate and reproducible positioning of a patient for treatment is often accomplished with the use of a thermoplastic mask. Such a mask often consists of a thermoplastic layer, often bearing multiple fenestrations, that is custom molded to the individual person's facial contours and head. Such a mask is often formed in two parts, a facial and an occipital part, with each part being secured to an often-U-shaped rigid frame in the form of an upper and lower frame. The patients may be placed such that the occipital portion cradles the head, while the facial portion is then overlaid on the face. This tends to bring opposing securing holes on the upper frame and lower frame into opposition, such that various type of closures may be inserted through both frames to hold the frames, and therefore the mask portions, in the correct alignment. The frames are then generally secured to an outside substrate, such as to a treatment table, for steady and repeatable patient positioning. Since patients' facial contours can change over time, particularly in response to treatment or medications, swelling not being uncommon, it is highly desirable to have a means of adjusting the opposition of the frames, so that the mask may be made slightly tighter or looser. This is most easily accomplished by closures that allow a variable separation between the frames.

### SUMMARY OF THE INVENTION

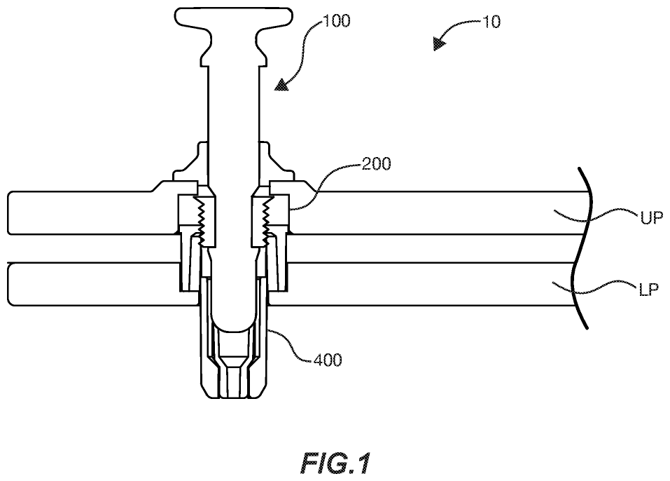

The disclosed invention relates to methods to for making a pin closure system. Realizing that one skilled in the art may appreciate multiple additional structures and methods that might be incorporated, the mode of operation of embodiments of the present invention may be broadly described, by way of example and not limitation only, as follows. As seen well in FIGS. 1-8, the system (10) may be used to reversibly secure an upper (UP) and a lower plate (LP), particularly the upper and lower frame plates of a radiation therapy mask, and then further to another substrate (S), such as a treatment board. In this specification, the term proximal shall mean those portions of the system (10) that are closer to a user's grip at a pin grip (110), relative to those portions which are further from a user's grip, which shall be deemed distal. One skilled in the art will realize that a plethora of materials may be useful in forming the system (10) but that a common material may be a thermoplastic having high strength and yet a degree of resilience.

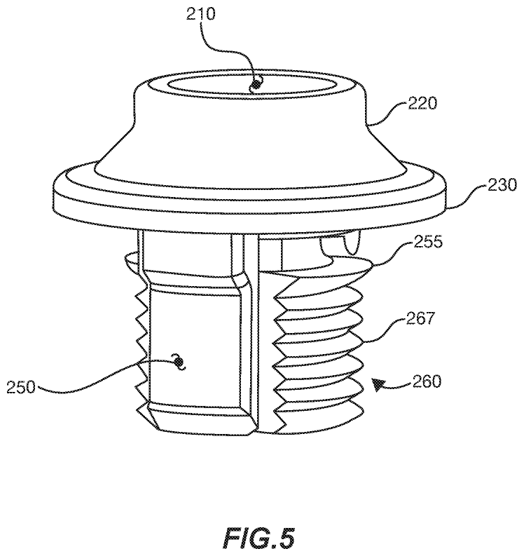

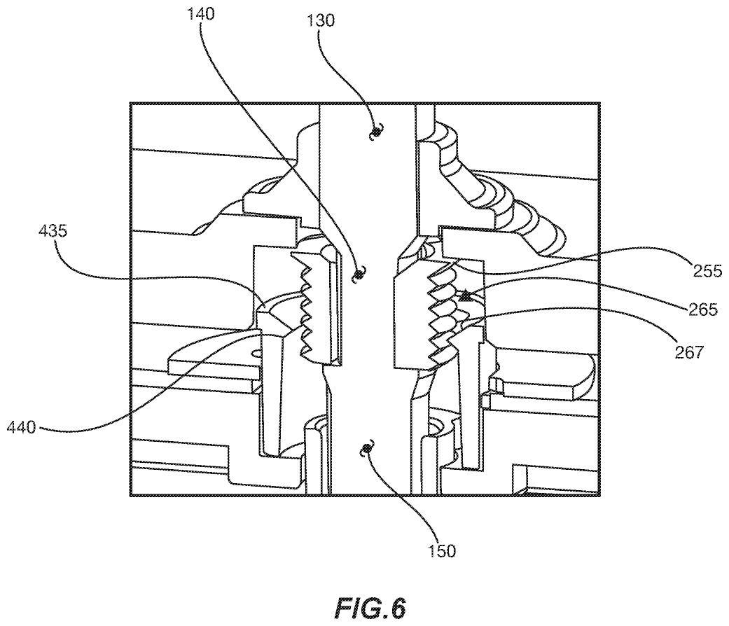

Assembly begins, as can be seen well in FIGS. 1 and 6, with placing the upper sleeve (200) in a proper orientation, aided by an upper sleeve orienter (250), within a shaped fenestration in the upper plate (UP). As the upper sleeve (200) passes through the fenestration, there is a slight compression of the upper sleeve expansile portion (260), as an upper sleeve to upper plate engager (255) at a proximal portion of the upper sleeve expansile portion (260) has a slightly larger diameter than that of the shaped fenestration in the upper plate (UP). Once the upper sleeve to upper plate engager (255) passes the upper plate (UP) fenestration, the upper sleeve expansile portion re-expands, as seen well in FIGS. 5 and 6, and the upper sleeve (200) become trapped in the upper plate (UP), held between the upper sleeve bearing collar (230) and the upper sleeve to upper plate engager (255), as seen well in FIGS. 5 and 6

A user may insert a pin (100) through the upper sleeve central fenestration (210) of an upper sleeve barrel portion (240) of an upper sleeve (200) by gripping the pin (100) by a pin grip (110) and axially inserting the pin (100) into the upper sleeve (200) in a proximal to distal direction. In those embodiments where the pin (100) has a pin distal diameter (150), a pin central diameter (140) and a pin proximal diameter (130), as the pin distal diameter (150) begins to traverse the upper sleeve expansile portion (260) the upper sleeve expansile portion (260) is expanded laterally, allowing passage of the pin distal diameter (150) through the upper sleeve expansile portion (260) at the distal portion of the upper sleeve (200).

At some point, the pin shelf (160), in those having same, may emerge from the upper sleeve expansile position (260), and again in those embodiments having same, the pin distal diameter (150) may transition to a smaller pin central diameter (140), thereby effectively trapping the pin distal diameter (150) and the pin shelf (160) (if present) distal to the upper sleeve expansile portion (260). Thus, to put it most simply, the pin (100) can no longer fall out of the upper sleeve (200), retrograde in a proximal direction, from the upper sleeve (200).

Contemporaneously, the lower sleeve (400), having a lower sleeve barrel portion (430) with a lower sleeve central fenestration (410) and a lower sleeve expansile portion (420) may be oriented to the lower plate by a lower sleeve orienter (450), which may be a lug, ridge or any other key-like structure, and be placed in a corresponding shaped fenestration on a distal aspect of a lower plate (LP), and engaged there by a lower sleeve to lower plate engager (460).

The full mask is then ready for assembly. A rear aspect of a patient's head may be placed in a molded enclosure attached to the lower plate (LP). A molded enclosure, generally custom molded to the patient's facial contour, and attached to an upper plate (UP) may then be applied, such that a plurality of corresponding fenestrations between the upper and lower plates (UP, LP) are in line with each other.

If needed or desired, one or more shims (300) may be placed between the upper plate (UP) and lower plate (LP) to vary the distance between the plates (UP. LP), and thus, vary the distance between the upper and lower sleeves (200, 400), and therefore make the mask a looser or tighter fit on the patient. In practice, this may mean making an initial fit to the patient with one or two shims (300), and then adding to or reducing the number of shims (300) as treatment progresses. This is often needed or desired as a result in changes in the patient's facial contour due to treatment or associated medications.

If the pin (100), which had previously been oriented such that the pin central diameter (140) was advanced so as to directly underly the upper sleeve expansile portion (260), the lower sleeve to upper sleeve engager (440) will loosely overlie the upper sleeve to lower sleeve engager (265). Since the system (10) is provided with one or more shims (300) that can vary the distance between the upper plate (UP) and the lower plate (LP) thus varying the distance between the upper sleeve (200) and the lower sleeve (400), one skilled in the art will see that a plurality of upper sleeve to lower sleeve engagers (265) will accommodate a varying distance between the upper plate (UP) and the lower plate (LP), and allow at least one upper sleeve to lower sleeve engager (265) on the upper sleeve expansile portion (260), to lie in close proximity to the upper sleeve to lower plate engager (440). Often, the upper sleeve to lower sleeve engagers (265) are presented as a plurality of parallel threads (267) arranged in a proximal to distal manner on the surface of the upper sleeve to lower sleeve engager (265) to accommodate the potential use of shims (300).

Advancement of the pin (100), such that the pin proximal diameter (130) underlies the upper sleeve expansile portion (260) will expand the upper sleeve expansile portion (260), thus causing the upper sleeve to lower sleeve engager (265) to become engaged with the lower sleeve to upper sleeve engager (440).

If the pin (100) is advanced further in a distal axial direction, the pin distal diameter (150) begins traversing the lower sleeve expansile portion (420) where the internal diameter of the lower sleeve central fenestration (410) may be less than the pin distal diameter (150). This may cause an outward expansion of the lower sleeve expansile portion (420). One skilled in the art will see that if the system (10) is overlaid on an appropriately shaped and sized fenestration in an external substrate, such as a treatment table or other substrate as seen in FIG. 1, where the unexpanded lower sleeve expansile portion (420) closely fits within the fenestration of the substrate, the expansion of the lower sleeve expansile portion (420) will effectively “lock” the system (10) to the substrate (S). Thus, the upper plate (UP), upper sleeve (200), lower sleeve (400), pin (100), and lower plate (LP) can all be simultaneously “locked” together, and even jointly all be “locked” to the substrate (S).

Again, one skilled in the art will realize that partially withdrawing the pin (100) such that the pin distal diameter (150) is retracted proximally to the lower sleeve expansile portion (420), the resilience of the lower sleeve (400) material will cause the lower sleeve expansile portion (420) to collapse, thus releasing the “lock” from the substrate, but wherein the pin proximal diameter (130), if still within the upper sleeve expansile portion (260), will maintain the connection between the upper and lower sleeves. Continuing to withdraw the pin (100) will cause the pin central diameter (140) to overlie the upper sleeve expansile portion (260) thus releasing the upper sleeve (200) and the lower sleeve (400). Such a release may be used to fully release the mask from the patient or may be utilized simply to loosen the upper plate (UP) and lower plate (LP), so that one or more shims (300) may be provided. One can also easily see how a shim (300) that is provided with a shim central fenestration (310) and shim lateral opening (320) can be easily added or removed, as they can be fit around the pin (100) without fully withdrawing the pin (100) from the upper plate (UP) and lower plate (LP). A slight enlargement of the lower sleeve barrel portion (430) at the lower sleeve bearing collar (435) may help prevent any shims (300) present from slipping off the lower sleeve barrel portion (430).

In many embodiments, a pin stop (120) may prevent the pin (100) from entering the upper sleeve central fenestration (210), and this, combined with the presence of an upper sleeve spacing collar (220) may preserve sufficient space distal to the pin grip (110) to make a user's grip more convenient and effective on the pin (100).

### BRIEF DESCRIPTION OF THE ILLUSTRATIONS

Without limiting the scope of the as disclosed herein and referring now to the drawings and figures:

FIG. 1 is a cross-section assembled view of an embodiment of the present invention;

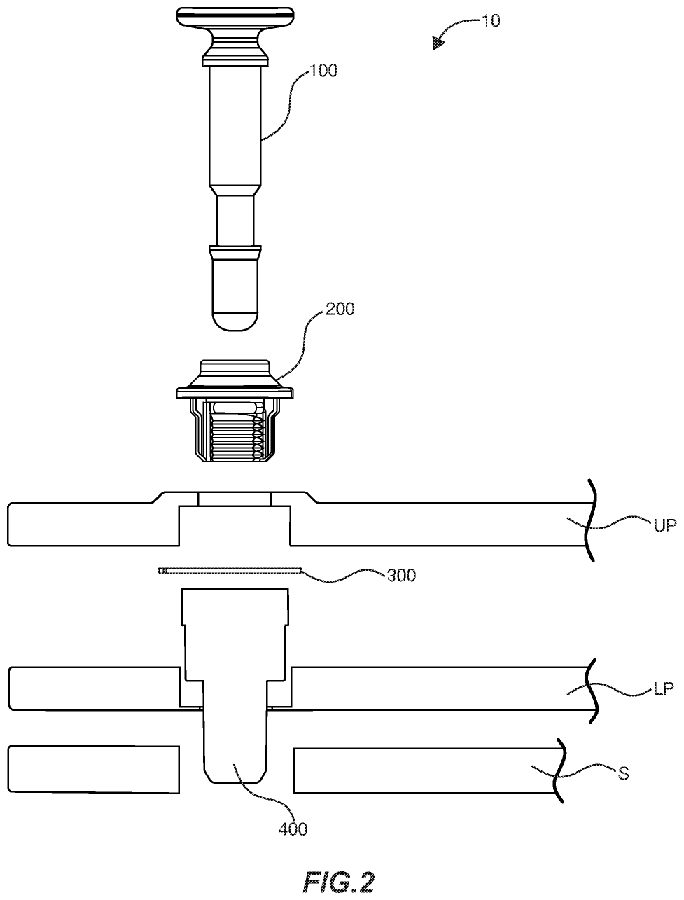

FIG. 2 is a cross-section exploded view of the embodiment of FIG. 1;

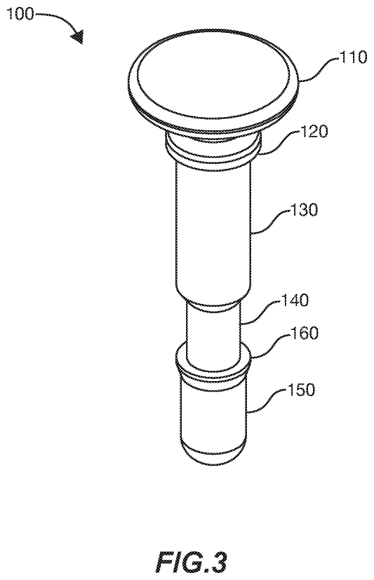

FIG. 3 is an elevated perspective view of a portion of the embodiment of FIG. 1;

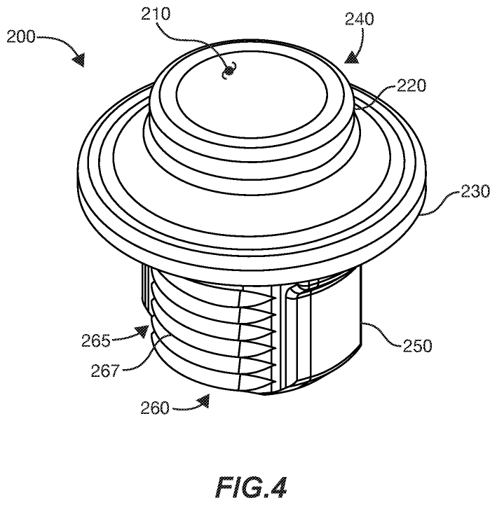

FIG. 4 is an elevated perspective view of another portion of the embodiment of FIG. 1;

FIG. 5 is an elevated perspective view of another portion of the embodiment of FIG. 1;

FIG. 6 is an elevated cross-section view of another portion of the embodiment of FIG. 1;

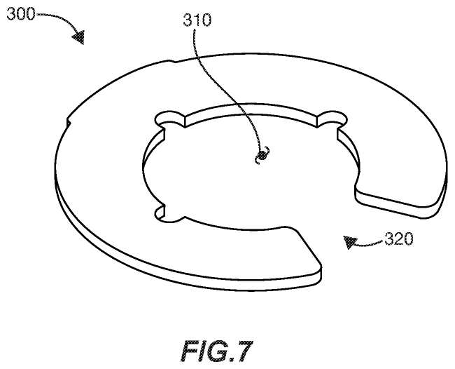

FIG. 7 is an elevated perspective view of another portion of the embodiment of FIG. 1; and

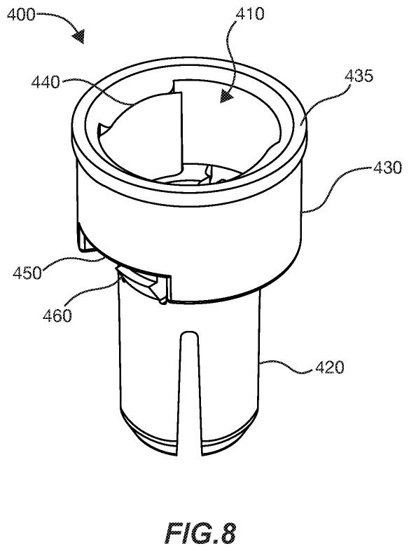

FIG. 8 is an elevated perspective view of another portion of the embodiment of FIG. 1.

These illustrations are provided to assist in the understanding of the exemplary embodiments of the method of forming a pin closure system and materials related thereto described in more detail below and should not be construed as unduly limiting the specification.

In particular, the relative spacing, positioning, sizing and dimensions of the various elements illustrated in the drawings may not be drawn to scale and may have been exaggerated, reduced or otherwise modified for the purpose of improved clarity. Those of ordinary skill in the art will also appreciate that a range of alternative configurations have been omitted simply to improve the clarity and reduce the number of drawings.

### DETAILED DESCRIPTION OF THE INVENTION

As seen well in FIGS. 1-8, the system (10) may be used to reversibly secure an upper (UP) and a lower plate (LP), particularly the upper and lower frame plates of a radiation therapy mask, and then further to another substrate (S), such as a treatment board, as seen well in FIGS. 1 and 2. In this specification, the term proximal shall mean those portions of the system (10) that are closer to a user's grip at a pin grip (110), relative to those portions which are further from a user's grip, which shall be deemed distal.

What is claimed then, is a pin closure system (10), having, among other components, a pin (100), with at least a pin grip (110) and at least one pin diameter (150). The system further includes an upper sleeve (200), seen well in FIGS. 2, 4, 5 and 6, having an upper sleeve barrel portion (240) and an upper sleeve central fenestration (210) reversibly receiving at least a portion of the pin (100). There may be an upper sleeve bearing collar (230) having an upper sleeve bearing collar surface (generally but not necessarily a lower surface of the bearing collar (230)) bearing against a surface of an upper plate (UP).

The upper sleeve may further have an upper sleeve orienter (250) orienting the upper sleeve (200) in a predetermined orientation within a shaped fenestration in the upper plate (UP) In some embodiments, there may be an upper sleeve to upper plate engager (255) engaging the upper sleeve (200) within the shaped fenestration of the upper plate (UP), and a reversibly radially expandable upper sleeve expansile portion (260) at a distal portion of the upper sleeve barrel (200) having a plurality of upper sleeve to lower sleeve engagers (265),

The system (10) may also include a lower sleeve (400), seen well in FIGS. 6 and 8, having a lower sleeve barrel portion (430) and a lower sleeve central fenestration (410) reversibly receiving at least a portion of the pin (100) and a lower sleeve bearing collar (435) having a lower sleeve bearing collar surface that may bear against the upper plate (UP). However, as seen well in FIG. 6, such bearing will depend on the depth at which the upper sleeve (US) and lower sleeve (LS) engage.

There may be a lower sleeve orienter (450) orienting the lower sleeve (400) in a predetermined orientation within a shaped fenestration in the lower plate (LP), and a lower sleeve to upper sleeve engager (440) engaging the lower sleeve (400) to the upper sleeve (200). In further embodiments, there may be at least one upper sleeve to lower sleeve engager (265), a lower sleeve to lower plate engager (460) engaging the lower sleeve (400) to the lower plate (LP) and a reversibly radially expandable lower sleeve expansile portion (420) at a distal portion of the lower sleeve barrel portion (430).

In yet other embodiments, as would be seen by one skilled in the art, the system (10) may be configured such that the at least one pin diameter (150) further includes a pin proximal diameter (130) a pin central diameter (140) and a pin distal diameter (150). In a subset of embodiments, the pin central diameter (140) may less that the pin proximal diameter (130).

In still further embodiments, at least one shim (300) may be placed between opposing surfaces of the upper plate (UP) and the lower plate (LP), thereby increasing a separation of the upper plate (UP) and the lower plate (LP). The shim (300) may have a shim central fenestration (310) and a shim lateral opening (320). In some embodiments, the at least one shim (300) may be a plurality of shims (300) and each shim (300) may be approximately one (1) millimeter thick.

In other embodiments, the pin (100) may have a pin stop (120), larger than the upper sleeve central fenestration (210) in a proximal aspect of the upper sleeve barrel portion (240) thereby limiting the axial travel of the pin (100) in a distal direction, while in yet others, the pin (100) may have a pin shelf (160) greater in diameter than the upper sleeve central fenestration (210) in an unexpanded upper sleeve expansile portion (260), thereby limiting the axial travel of the pin (100) in a proximal direction, proximal to said unexpanded upper sleeve expansile portion (260). The pin shelf (160) diameter, in some embodiments, may be approximately equal to the pin proximal diameter (130).

In some embodiments of the present invention, the upper sleeve (200) may have an upper sleeve spacing collar (220) separating the pin grip (110) and the upper sleeve bearing collar (230), thereby creating space for a user's fingers to grip the pin grip (110). In yet further embodiments, the upper sleeve to lower sleeve engager (265) may further include a plurality of raised threads (267).

In a significant advance, the system may also be used to secure the upper plate (UP) and lower plate (LP) to an external substrate (S) such as a treatment table. This may, among numerous possibilities, be accomplished by having the lower sleeve expansile portion (420) extending at least partially into a fenestration in an external substrate (S) below the lower plate (LP). The system (10) may be reversibly coupled to the external substrate (S) by expansion of the lower sleeve expansile portion (420) within a fenestration in the external substrate. Thus, a single pin closure may simultaneously engage the upper and lower plates of a radiation therapy mask and hold that mask in position on an external modality, such as a treatment table, to accomplish solid and easily reproducible positioning.

In yet other embodiments, a pin closure system (10), may have, among other components, a pin (100), with at least a pin grip (110) and at least one pin diameter (150). The system further includes an upper sleeve (200), seen well in FIGS. 2, 4, 5 and 6, having an upper sleeve barrel portion (240) and an upper sleeve central fenestration (210) reversibly receiving at least a portion of the pin (100). There may be an upper sleeve bearing collar (230) having an upper sleeve bearing collar surface (generally but not necessarily a lower surface of the bearing collar (230)) bearing against a surface of an upper plate (UP).

The upper sleeve may further have an upper sleeve orienter (250) orienting the upper sleeve (200) in a predetermined orientation within a shaped fenestration in the upper plate (UP) In some embodiments, there may be an upper sleeve to upper plate engager (255) engaging the upper sleeve (200) within the shaped fenestration of the upper plate (UP), and a reversibly radially expandable upper sleeve expansile portion (260) at a distal portion of the upper sleeve barrel (200) having a plurality of upper sleeve to lower sleeve engagers (265),

The system (10) may also include a lower sleeve (400), seen well in FIGS. 6 and 8, having a lower sleeve barrel portion (430) and a lower sleeve central fenestration (410) reversibly receiving at least a portion of the pin (100) and a lower sleeve bearing collar (435) having a lower sleeve bearing collar surface that may bear against the upper plate (UP). However, as seen well in FIG. 6, such bearing will depend on the depth at which the upper sleeve (US) and lower sleeve (LS) engage.

There may be a lower sleeve orienter (450) orienting the lower sleeve (400) in a predetermined orientation within a shaped fenestration in the lower plate (LP), and a lower sleeve to upper sleeve engager (440) engaging the lower sleeve (400) to the upper sleeve (200). In further embodiments, there may be at least one upper sleeve to lower sleeve engager (265) formed by a plurality of raised threads (267), a lower sleeve to lower plate engager (460) engaging the lower sleeve (400) to the lower plate (LP) and a reversibly radially expandable lower sleeve expansile portion (420) at a distal portion of the lower sleeve barrel portion (430).

In further embodiments, at least one shim (300) may be placed between opposing surfaces of the upper plate (UP) and the lower plate (LP), thereby increasing a separation of the upper plate (UP) and the lower plate (LP).

As previously indicated above, the system (10) may be reversibly coupled to an external substrate (S), and this may be accomplished by expansion of the lower sleeve expansile portion (420) within a fenestration in the external substrate (S).

One skilled in the art will envision many ways that the system may be assembled and used. These may include the steps of first placing and engaging an upper sleeve (200) having an upper sleeve central fenestration (210) and an upper sleeve expansile portion (260) in a shaped fenestration in an upper plate (UP). This may be followed by placing and engaging a lower sleeve (400) having a lower sleeve central fenestration (410) and a lower sleeve expansile portion (420) in a shaped fenestration of a lower plate (LP).

A further step might include opposing the shaped fenestrations in the upper plate (UP) and lower plate (LP), and the inserting, in an axial direction, a pin (100) through the upper sleeve central fenestration (210) and the lower sleeve central fenestration (410). This may cause expansion the upper sleeve expansile portion (260) and the lower sleeve expansile portion (420), thereby coupling the upper sleeve (200), the lower sleeve (400), the upper plate (UP) and the lower plate (LP).

A further set of steps could include the step of inserting at least a portion of the lower sleeve (400) within a fenestration of an external substrate, and expanding at least a portion of the lower sleeve (400), thereby securing the upper sleeve (200), the lower sleeve (400), the upper plate (UP) and the lower plate (LP) to the external substrate (S).

Numerous alterations, modifications, and variations of the preferred embodiments disclosed herein will be apparent to those skilled in the art and they are all anticipated and contemplated to be within the spirit and scope of the disclosed specification. For example, although specific embodiments have been described in detail, those with skill in the art will understand that the preceding embodiments and variations can be modified to incorporate various types of substitute and or additional or alternative materials, relative arrangement of elements, order of steps and additional steps, and dimensional configurations. Accordingly, even though only few variations of the method and products are described herein, it is to be understood that the practice of such additional modifications and variations and the equivalents thereof, are within the spirit and scope of the method and products as defined in the following claims. The corresponding structures, materials, acts, and equivalents of all means or step plus function elements in the claims below are intended to include any structure, material, or acts for performing the functions in combination with other claimed elements as specifically claimed.

I claim:

1. A pin closure system (10), comprising:
a pin (100), having at least a pin grip (110) and at least one pin diameter (150),
an upper sleeve (200), having an upper sleeve barrel portion (240) and a upper sleeve central fenestration (210) reversibly receiving at least a portion of the pin (100), an upper sleeve bearing collar (230) having an upper sleeve bearing collar surface bearing against a surface of an upper plate (UP), an upper sleeve orienter (250) orienting the upper sleeve (200) in a predetermined orientation within a shaped fenestration in the upper plate (UP), an upper sleeve to upper plate engager (255) engaging the upper sleeve (200) within the shaped fenestration of the upper plate (UP), and a reversibly radially expandable upper sleeve expansile portion (260) at a distal portion of the upper sleeve barrel (200) having a plurality of upper sleeve to lower sleeve engagers (265), and
a lower sleeve (400), having a lower sleeve barrel portion (430) and a lower sleeve central fenestration (410) reversibly receiving at least a portion of the pin (100), a lower sleeve bearing collar (435) having a lower sleeve bearing collar surface bearing against the surface of the upper sleeve to upper plate engager (255), a lower sleeve orienter (450) orienting the lower sleeve (400) in a predetermined orientation within a shaped fenestration in the lower plate (LP), a lower sleeve to upper sleeve engager (440) engaging the lower sleeve (400) to the upper sleeve (200), a lower sleeve to lower plate engager (460) engaging the lower sleeve (400) to the lower plate (LP) and a reversibly radially expandable lower sleeve expansile portion (420) at a distal portion of the lower sleeve barrel portion (430).

2. The system (10) according to claim 1, wherein the at least one pin diameter (150) further comprises a pin proximal diameter (130), a pin central diameter (140) and a pin distal diameter (150).

3. The system (10) according to claim 2, wherein the pin central diameter (140) is less that the pin proximal diameter (130).

5. The system (10) according to claim 1, wherein at least one shim (300) is placed between opposing surfaces of the upper plate (UP) and the lower plate (LP), thereby increasing a separation of the upper plate (UP) and the lower plate (LP).

6. The system (10) according to claim 5, wherein the shim (300) has a shim central fenestration (310) and a shim lateral opening (320).

7. The system (10) according to claim 5, wherein the shim (300) is approximately one (1) millimeter thick.

8. The system (10) according to claim 5, wherein the at least one shim (300) is a plurality of shims (300).

9. The system (10) according to claim 1, wherein the pin (100) has a pin stop (120), larger than the upper sleeve central fenestration (210) in a proximal aspect of the upper sleeve barrel portion (240) thereby limiting the axial travel of the pin (100) in a distal direction.

10. The system (10) according to claim 1, wherein the pin (100) has a pin shelf (160) greater in diameter than the upper sleeve central fenestration (210) in an unexpanded upper sleeve expansile portion (260), thereby limiting the axial travel of the pin (100) in a proximal direction, proximal to said unexpanded upper sleeve expansile portion (260).

11. The system (10) according to claim 10, wherein a pin shelf (160) diameter is approximately equal to the pin proximal diameter (130).

12. The system (10) according to claim 1, wherein the upper sleeve (200) has an upper sleeve spacing collar (220) separating the pin grip (110) and the upper sleeve bearing collar (230), thereby creating space for a user's fingers to grip the pin grip (110).

13. The system (10) according to claim 1, wherein the upper sleeve to lower sleeve engager (265) further comprises a plurality of raised threads (267).

14. The system (10) according to claim 1, wherein the system is reversibly coupled to an external substrate (S).

15. The system (10) according to claim 13, wherein the external substrate (S) is a treatment table.

16. A pin closure system (10), comprising:
a pin (100), having at least a pin grip (110) and at least one pin diameter (150), an upper sleeve (200), having an upper sleeve barrel portion (240) and a upper sleeve central fenestration (210) reversibly receiving at least a portion of the pin (100), an upper sleeve bearing collar (230) having an upper sleeve bearing collar surface bearing against a surface of an upper plate (UP), an upper sleeve orienter (250) orienting the upper sleeve (200) in a predetermined orientation within a shaped fenestration in the upper plate (UP), an upper sleeve to upper plate engager (255) engaging the upper sleeve (200) within the shaped fenestration of the upper plate (UP), and a reversibly radially expandable upper sleeve expansile portion (260) at a distal portion of the upper sleeve barrel (200) having a plurality of upper sleeve to lower sleeve engagers (265),
a lower sleeve (400), having a lower sleeve barrel portion (430) and a lower sleeve central fenestration (410) reversibly receiving at least a portion of the pin (100), a lower sleeve bearing collar (435) having a lower sleeve bearing collar surface that may bear against the upper plate (UP), a lower sleeve orienter (450) orienting the lower sleeve (400) in a predetermined orientation within a shaped fenestration in the lower plate (LP), a lower sleeve to upper sleeve engager (440) engaging the lower sleeve (400) to the upper sleeve (200) at least one upper sleeve to lower sleeve engager (265) comprised of a plurality of raised threads (267), a lower sleeve to lower plate engager (460) engaging the lower sleeve (400) to the lower plate (LP) and a reversibly radially expandable lower sleeve expansile portion (420) at a distal portion of the lower sleeve barrel portion (430).

17. The system according to claim 16, wherein at least one shim (300) is placed between opposing surfaces of the upper plate (UP) and the lower plate (LP), thereby increasing a separation of the upper plate (UP) and the lower plate (LP).

18. The system (10) according to claim 16, wherein the system (10) is reversibly coupled to an external substrate (S) by expansion of the lower sleeve expansile portion (420) within a fenestration in the external substrate (S).

19. A method for using a pin closure system (10) to engage an upper plate (UP) and a lower plate (LP) comprising the steps of:
a) placing and engaging an upper sleeve (200) having an upper sleeve central fenestration (210) and an upper sleeve expansile portion (260) in a shaped fenestration in an upper plate (UP),
b) placing and engaging a lower sleeve (400) having a lower sleeve central fenestration (410) and a lower sleeve expansile portion (420) in a shaped fenestration of a lower plate (LP),
c) opposing the shaped fenestrations in the upper plate (UP) and lower plate (LP), and
d) inserting, in an axial direction, a pin (100) through the upper sleeve central fenestration (210) and the lower sleeve central fenestration (410), thereby expanding the upper sleeve expansile portion (260) and the lower sleeve expansile portion (420), thereby coupling the upper sleeve (200), the lower sleeve (400), the upper plate (UP) and the lower plate (LP).

20. The method according to claim 19, further comprising the step of inserting at least a portion of the lower sleeve (400) within a fenestration of an external substrate (S), and expanding at least a portion of the lower sleeve (400), thereby securing the upper sleeve (200), the lower sleeve (400), the upper plate (UP) and the lower plate (LP) to the external substrate (S).
